(19) United States
(12) Patent Application Publication
(10) Pub. No.: US 2007/0016216 A1
(43) Pub. Date: Jan. 18, 2007

(54) Delivery System Having A Nozzle With An Adjustable Spacer And Method Of Use Thereof For Improving Line-Of-Sight During A Medical Procedure And Pressurizer For Improving Line-Of-Sight During A Medical Procedure

(76) Inventors: Christopher M. Tague, Delton, MI (US); Carlos Negroni, San Juan, PR (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 11/381,255

(22) Filed: May 2, 2006

Related U.S. Application Data
(60) Provisional application No. 60/676,773, filed on May 2, 2005.

(51) Int. Cl. A61F 2/00

(52) U.S. Cl. 606/94

(57) ABSTRACT

The present invention provides a delivery system for use with a pressurizer to deliver a flowable material such as bone cement into an anatomical site under pressure. The delivery system includes a cartridge for holding the bone cement. A nozzle is mounted to the cartridge to convey the bone cement to the anatomical site. A spacer is movable along the nozzle between a plurality of adjustment positions to provide a variable clearance distance between the cartridge and the pressurizer to maintain a line-of-sight to the pressurizer and the anatomical site. The line-of-sight is particularly beneficial when using the delivery system in minimally invasive surgical procedures. A method of delivering the bone cement and an alternative pressurizer are also provided.

### CROSS REFERENCE TO RELATED APPLICATION

This application claims the benefit of U.S. provisional patent application Ser. No. 60/676,773 filed May 2, 2005, the advantages and disclosure of which are hereby incorporated by reference.

### FIELD OF THE INVENTION

The present invention generally relates to a delivery system for use with a pressurizer to deliver a flowable material into an anatomical site under pressure during a medical procedure. More specifically, the present invention relates to a nozzle of the delivery system for conveying the flowable material to the anatomical site under pressure during minimally invasive surgical procedures. The present invention also relates to pressurizers for pressurizing the flowable material.

### BACKGROUND OF THE INVENTION

Flowable materials that are capable of setting to a hardened condition, such as bone cement, are well known for anchoring an implant, such as a hip or shoulder implant, in a bone opening or bone canal. Typically, the bone cement is prepared in a bone cement mixer and then transferred to a delivery system. Alternatively, the bone cement can be mixed directly in a bone cement cartridge that is then used in the delivery system. The delivery system typically includes a nozzle to convey the bone cement into the bone canal.

In a typical medical procedure, a medical professional, e.g., a surgeon or surgeon's assistant, exposes a patient's bone by making an incision and folding the tissue back to expose the bone. The bone opening or bone canal is formed in the bone for the receipt of the bone cement and the implant to be anchored. In one technique, such as during total hip arthroplasty procedures, the nozzle is inserted directly into the bone canal and the bone canal is filled with bone cement by ejecting the bone cement from the nozzle into the bone canal.

In a further step of this technique, a pressurizer is used to apply pressure to the bone cement so that the bone cement is forced into small openings in the bone. The pressurizer is placed on the nozzle and then pressed against the bone in the area of the bone canal. Pressure is applied to the pressurizer to hold the pressurizer against the bone when delivering the bone cement into the bone canal. The pressurizer allows the bone cement to be delivered under pressure so that the bone cement not only fills the bone canal, but also enters the small openings of the bone to reduce or eliminate air pockets in the bone cement and to aid in retention of the implant. Typically, the nozzle is attached to a container holding the bone cement and a distal end of the container is pressed against the pressurizer to provide the seal between the pressurizer and the bone to develop the necessary pressure within the bone canal.

In this type of procedure, with the tissue folded back, the surgeon has a fairly large exposed area to form the bone canal in the bone and manipulate the delivery system, including the nozzle. The exposed work area is also large enough to provide the surgeon with a line-of-sight to easily see the exposed bone and the nozzle. Due to the large work area, a distal end of the container can directly engage the pressurizer to create the seal and necessary pressure.

A new procedure that is gaining wide acceptance by medical professionals has been developed for anchoring implants in bone. This new procedure is a minimally invasive surgical procedure that employs a smaller incision to expose the bone. Minimally invasive surgery (MIS) allows the surgeon to perform medical procedures, such as total hip arthroplasty, through one or two smaller incisions. A single minimally invasive incision may measure only 3 to 6 inches in length. In total hip arthroplasty, if two incisions are employed, a 2 to 3 inch incision is made over the groin for placement of a socket and a 1 to 2 inch incision is made over the buttock for placement of a stem. Benefits of MIS may include less damage to surrounding tissues, faster rehabilitation, and shorter hospital time. With the smaller incision(s), the tissue cannot be folded back, resulting in a much smaller work area. The smaller work area makes it more difficult to get the nozzle in the proper position and also reduces the surgeon's line-of-sight. Additionally, with larger patients, the patient's flesh inhibits the surgeon from not only positioning the nozzle in the bone canal, but also from applying pressure to the container that normally would abut the pressurizer. The patient's flesh obstructs the surgeon's view resulting in an inability to press the container against the pressurizer.

For MIS, as well as for improving existing procedures, there is a need in the art to provide a delivery system or pressurizer that improves the surgeon's line-of-sight to the work area, i.e. the bone canal, to properly apply pressure to the pressurizer and pressurize the bone cement in the bone canal.

### SUMMARY OF THE INVENTION AND ADVANTAGES

The present invention provides a delivery system for use with a pressurizer to deliver a flowable material into an anatomical site during a medical procedure. The delivery system includes a container for holding the flowable material. A nozzle includes a proximal end operatively coupled to the container and a distal end spaced from the proximal end for conveying the flowable material to the anatomical site. A spacer is adjustable along the nozzle between a plurality of adjustment positions to provide a variable clearance distance between the container and the pressurizer to maintain a line-of-sight to the pressurizer and the anatomical site during the medical procedure.

In another aspect of the present invention, the nozzle includes a nozzle body and the spacer is adjustable along the nozzle body between the plurality of adjustment positions to provide the variable clearance distance between the container and the pressurizer.

A method of delivering the flowable material to the anatomical site under pressure using the delivery system is also provided. The method includes sealing the pressurizer at the anatomical site by pressing the pressurizer against the anatomical site with the spacer of the nozzle. The flowable material is then delivered to the anatomical site under pressure. Prior to sealing the pressurizer, the spacer of the nozzle is adjusted between the plurality of adjustment positions to provide the clearance distance between the container and the pressurizer to maintain a line-of-sight to the pressurizer and the anatomical site during the medical procedure.

A pressurizer for use with a delivery system having a container for holding a flowable material and a nozzle operatively coupled to the container to convey the flowable material into the anatomical site under pressure is also provided. The pressurizer includes a body of pliable material defining a channel for receiving the nozzle and an outlet for providing communication between the nozzle and the anatomical site. The body has an outer surface for sealing against the anatomical site and a maximum width measured generally transverse to the channel. The width is less than 3 inches to accommodate MIS by facilitating insertion of the pressurizer into a minimally invasive incision, which generally measures from 3 to 6 inches in length. A neck of rigid material extends from the body to an end spaced from the body to provide a clearance distance between the container and the body to maintain a line-of-sight to the body and the anatomical site during the medical procedure.

The delivery system, method of use, and the pressurizer of the present invention, improve a medical professional's line-of-sight to the anatomical site of interest during a medical procedure, particularly, but not limited to, a minimally invasive surgical procedure. For instance, by providing the spacer that spaces the container of the delivery system from the pressurizer, the line-of-sight can be improved. Also, by providing for the adjustment of the spacer, the line-of-sight can be adjusted to a user's preference. Additionally, by providing the neck that extends away from the body on the pressurizer, the line-of-sight can be further improved. Improving the line-of-sight assists in ensuring that an appropriate seal is made about the anatomical site with the pressurizer thereby improving pressurization of the flowable material in the anatomical site and increasing the life of implants that are anchored in the anatomical site.

### BRIEF DESCRIPTION OF THE DRAWINGS

Other advantages of the present invention will be readily appreciated, as the same becomes better understood by reference to the following detailed description when considered in connection with the accompanying drawings wherein:

FIG. 1A is a perspective view of a delivery system of the present invention being used to deliver a flowable material such as bone cement into a prepared bone canal;

FIG. 1B is a perspective view of the delivery system of the present invention being used with a pressurizer to deliver a flowable material such as bone cement into the bone canal under pressure;

FIG. 2 is a cross-sectional view of a nozzle of the delivery system with a spacer adjustable along the nozzle;

FIG. 3 is a side elevational view of an alternative nozzle of the present invention with a flexible conduit;

FIG. 4 is a cross-sectional view of another alternative nozzle of the present invention with a flared distal end portion;

FIG. 5 is a side elevational view of an alternative delivery system of the present invention with a delivery gun for supporting a container and nozzle and an adjustment mechanism for adjusting a spacer along the nozzle;

FIG. 6 is a front elevational view of the spacer and the adjustment mechanism of FIG. 5;

FIG. 7 is a side elevational view of an alternative nozzle having an integrally formed spacer;

FIG. 8 is a cross-sectional view of the alternative nozzle of FIG. 7;

FIG. 9 is a side elevational view of the alternative nozzle assembly of FIG. 7 being used with a pressurizer to pressurize bone cement in a bone canal;

FIG. 10 is a perpective view of an alternative pressurizer of the present invention;

FIG. 11 is a cross-sectional view of the pressurizer of FIG. 10;

FIG. 12 is a top view of the pressurizer of FIG. 10;

FIG. 13 is a bottom view of the pressurizer of FIG. 10; and

FIG. 14 is a cross-sectional view of another alternative pressurizer of the present invention.

### DETAILED DESCRIPTION OF THE INVENTION

Referring to the Figures, wherein like numerals indicate like or corresponding parts throughout the several views, a delivery system for use with a pressurizer 22 to deliver a flowable material to an anatomical site 24 is generally shown at 20. The flowable material may be any material suitable for use in medical procedures, but preferably includes flowable materials that are capable of setting to a hardened condition, particularly those materials suited for anchoring implants in surgically-prepared bone openings or bone canals. Such a material includes bone cement, which may be formed by mixing bone cement powder (e.g., polymethylmethacrylate “PMMA”) and liquid monomer (e.g., methyl methacrylate monomer) to form a uniform bone cement mixture. For purposes of description, the flowable material shall be hereinafter described as the bone cement 26.

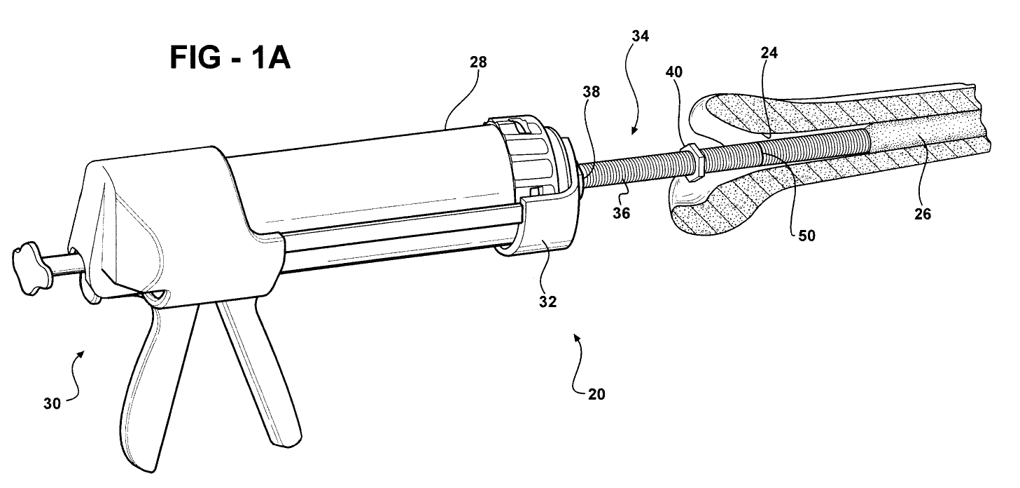

Referring to FIGS. 1A and 1B, the delivery system 20 includes a container 28 for holding the bone cement 26. The container 28 may be a cartridge 28 of a bone cement mixing and delivery system such as that shown in U.S. Patent Application Publication Nos. 2004/0267272 and 2005/0128867, both to Henniges et al., and both hereby incorporated by reference. In this type of system, a delivery gun 30 supports the cartridge 28 in a cradle 32 and drives the bone cement 26 from the cartridge 28 to the anatomical site 24, such as a prepared bone opening or bone canal 24 in bone. In FIGS. 1A and 1B, the anatomical site 24 is a surgically-prepared medullary canal 24 in a femur.

A nozzle 34 is mounted to the cartridge 28 to convey the bone cement 26 from the cartridge 28 to the bone canal 24. More specifically, the nozzle 34 includes a proximal end operatively coupled to the cartridge 28 and a distal end spaced from the proximal end to convey the flowable material to the bone canal 24. The nozzle 34 is generally tubular and has a nozzle body 36 for insertion into the bone canal 24 and a connector 38 for connecting the proximal end of the nozzle 34 to the cartridge 28. The connector 38 may include any suitable mechanism for coupling the nozzle 34 to the cartridge 28 such as the connector shown in U.S. Patent Application Publication Nos. 2004/0267272 and 2005/0128867, both of which are hereby incorporated by reference. The nozzle 34 is preferably formed from a biocompatible material such as, for example, a medical grade polymer, e.g., polycarbonate, polyethylene, or the like.

FIG. 1A illustrates the nozzle body 36 inserted well into the bone canal 24 to fill the bone canal 24 with the bone cement 26 using a well-known retrograde filling technique. FIG. 1B illustrates the nozzle body 36 after breaking a portion of the nozzle 34 off at a frangible section 50, as described below, and further illustrates use of the nozzle 34 with the pressurizer 22. Any conventional pressurizer 22 suitable for receiving the nozzle 34 may be used with the delivery system 20 of the present invention. Examples of suitable pressurizers are shown in U.S. Pat. No. 4,896,662 to Noble and U.S. Patent Application Publication No. 2005/0143748 to Negroni et al., both of which are hereby incorporated by reference.

Referring specifically to FIG. 1B, a spacer 40 is movable along the nozzle 34 to provide a variable clearance distance 42 between the cartridge 28 and the pressurizer 22 to maintain a line-of-sight to the pressurizer 22 and the bone. This spacer 40 is particularly useful in minimally invasive surgery (MIS) in which one or more incisions of generally less than 6 inches in length may be made to provide access to the bone canal 24. The present invention may be used in other types of surgical procedures as well, but is particularly adapted for use in minimally invasive surgical procedures. A maximum outer dimension of the spacer 40, measured generally transverse to the nozzle body 36, is preferably less than 6 inches, more preferably less than 3 inches, and most preferably less than 1 inch to facilitate entry of the spacer 40 into the incision(s) made in a patient during MIS.

An adjustment mechanism operatively engages the spacer 40 to adjust the spacer 40 between a plurality of adjustment positions along the nozzle 34. As a result, the user, such as the surgeon, can set the spacer 40 at a preferred adjustment position to provide the desired line-of-sight to the pressurizer 22 and the bone or the bone canal 24. This allows the user to easily adjust the length of the variable clearance distance 42 between the cartridge 28 and the pressurizer 22 to improve his or her ability to see the work area. In the embodiments of FIGS. 1A, 1B, and 2-4, the adjustment mechanism includes a plurality of threads 44 disposed along the nozzle 34 and the spacer 40 defines a threaded bore 46 for engaging the plurality of threads 44.

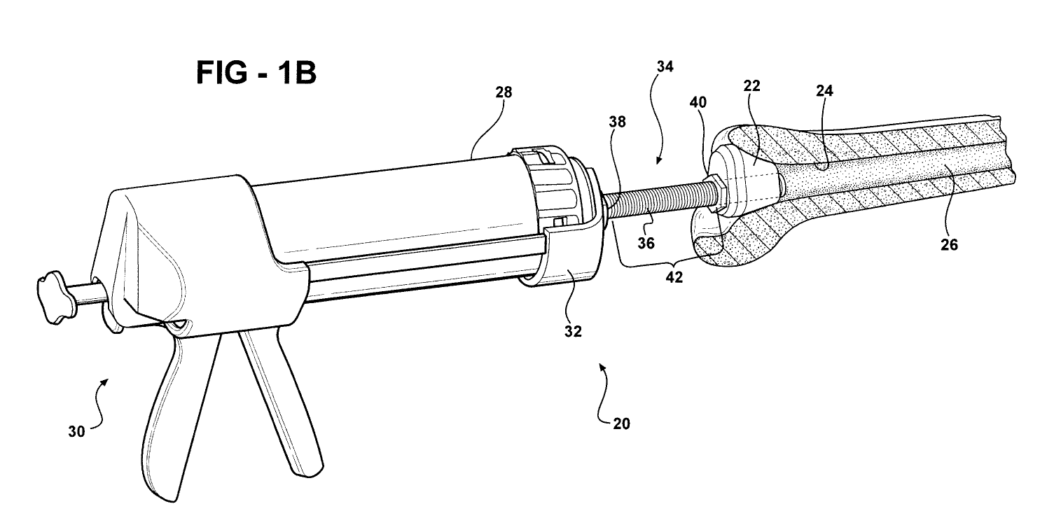

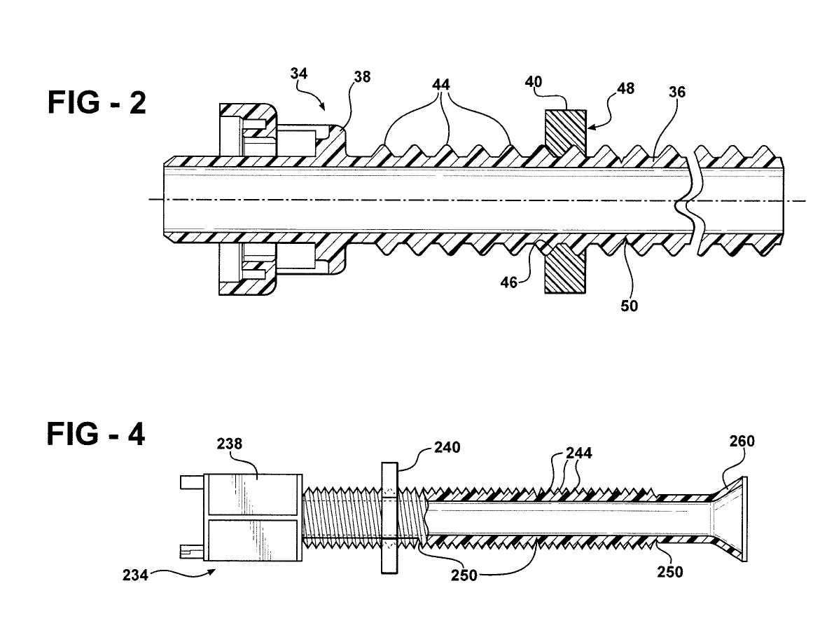

Referring specifically to FIG. 2, the nozzle 34 of FIGS. 1A and 1B is shown in more detail. The threads 44 are either integrally formed on the nozzle body 36 or are formed on a sleeve (not shown) that can be fitted over the nozzle body 36 and fixed to the nozzle body 36. The spacer 40 can be adjusted to the preferred adjustment position for engaging the pressurizer 22 by rotating relative to the nozzle body 36. A face 48 of the spacer 40 is adapted to engage the pressurizer 22 to allow pressurization to be completed. The spacer 40 is preferably formed from a biocompatible material such as, for example, a medical grade polymer or metal material, e.g., polycarbonate, polyethylene, stainless steel, or the like. A sealing material such as, for example, an elastomeric material, e.g., silicon rubber, foam, or the like, may be fixed to the face 48 of the spacer 40 by an adhesive or may be molded to the spacer 40 to provide a better seal between the spacer 40 and the pressurizer 22. A frangible section 50 is provided at a spaced distance from the distal end of the nozzle body 36 to allow a portion of the nozzle body 36 to be broken away to shorten the nozzle 34 to accommodate different surgical conditions.

In use, a medical professional, e.g., a surgeon or a surgeon's assistant, mixes the bone cement 26 either in the cartridge 28 or in a bone cement mixer. In the event a bone cement mixer is used, the bone cement 26, once mixed, is then transferred to the cartridge 28. Once mixing is complete, and the bone cement 26 is in the cartridge 28, the nozzle 34 is attached to a distal end of the cartridge 28. The surgeon can then use the retrograde filling technique, as shown in FIG. 1A, to fill the bone canal 24 with the bone cement 26. In this technique, the surgeon typically begins at the bottom of the bone canal 24, and as the bone canal 24 is filled, slowly withdraws the nozzle 34. Once filled, the nozzle 34 is broken at the frangible section 50. The pressurizer 22 is then inserted onto the nozzle 34 and the pressurizer 22 is then pressed against the bone in the area of the bone canal 24. The surgeon then engages the face 48 of the spacer 40 against the pressurizer 22 to apply pressure to the pressurizer 22 to seal the pressurizer 22 against the bone and allow pressurization of the bone cement 26 in the bone canal 24.

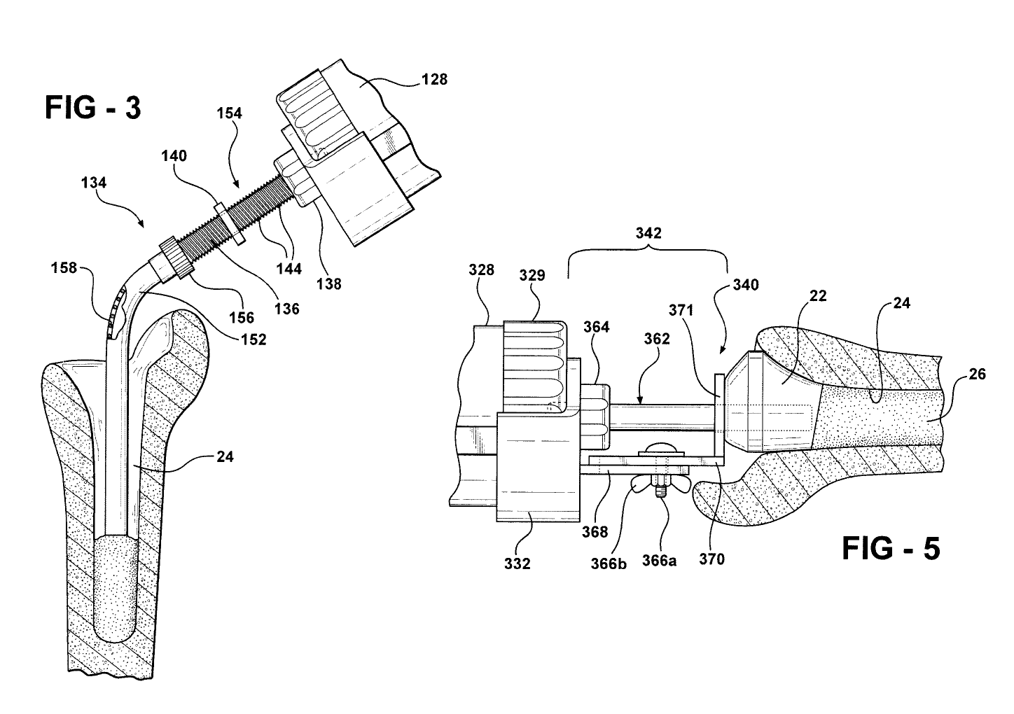

Referring to FIG. 3, an alternative nozzle 134 is shown. The same elements have been marked with the same numerals and like elements have been marked with similar numerals increased by 100. In this embodiment, the nozzle 134 includes a flexible conduit 152 extending to the distal end of the nozzle 134. The nozzle body 136 includes a rigid section 154, similar to the nozzle 134 of FIG. 2, and the flexible conduit 152 is coupled to the rigid section 154 with a threaded collar 156 or a releasable clamp (not shown) such as a hose clamp or the like. The flexible conduit 152 may be formed of medical grade silicone tubing, medical grade PVC tubing, or the like. The flexible conduit 152 may include a reinforcing coil 158 embedded in a wall of the flexible conduit 152 to reinforce the tubing of the flexible conduit 152.

In use, the flexible conduit 152 is inserted into the bone canal 24 of the bone for retrograde filling of the bone canal 24. The flexible conduit 152 allows the surgeon to bend the nozzle 134 to provide an offset of the cartridge 128 and nozzle 134 relative to the bone canal 24 thereby improving the line-of-site to the bone canal 124 and allowing the surgeon freedom of movement to adjust his or her line-of-sight to the bone canal 124. Once retrograde filling is complete, then the flexible conduit 152 can be removed from the rigid section 154 and the rigid section 154 can be used with the pressurizer 22, as shown in FIG. 1B.

Referring to FIG. 4, another alternative nozzle 234 is shown. In this embodiment, the same elements have been marked with the same numerals and like elements have been marked with similar numerals increased by 200. The nozzle 234 of FIG. 4 has a flared, generally cone shaped portion 260 at the distal end to facilitate the filling of the bone canal 24 with the bone cement 26. The flared portion 260 acts as a pressurizing device allowing pressurization of the bone cement 26 as the retrograde technique is being performed. In addition, this embodiment of the nozzle 234 has a plurality of frangible sections 250 for providing multiple lengths of the nozzle 234 during use. By breaking the nozzle 234 at any one of the frangible sections 250, the pressurizer 22 can be positioned over the nozzle 234 and used to further pressurize the bone cement 26 in the bone canal 24.

Referring to FIGS. 5-6, a further embodiment of the present invention is illustrated. In this embodiment, the same elements have been marked with the same numerals and like elements have been marked with similar numerals increased by 300. In this embodiment, a standard nozzle 362 is connected to the cartridge 328 by use of a standard nozzle connector 364. As illustrated, the cartridge 328 has a lid 329, which includes a male threaded nipple that is adapted to receive the mating female threaded member on the standard nozzle connector 364. It should be appreciated by those of ordinary skill in this art that other connectors could be used. For example, the threads could be reversed, a bayonet fastener could be used, etc. The cartridge 328 is inserted into the cradle 332 of the delivery gun 330.

In this embodiment, the delivery gun 330 provides the spacer 340 that allows adjustment of the line-of-sight for the surgeon. The spacer 340 comprises a base member 368 and a carrier member 370. The carrier member 370 has an end plate 371 defining a generally U-shaped opening 372 or slot (see FIG. 6). In this way, the cartridge 328 can be inserted into the delivery gun 330, with the standard nozzle 362 received within the U-shaped opening 372. In this embodiment, the adjustment mechanism includes a fastener 366a, 366b coupling the carrier member 370 to the base member 368 via elongated slots formed in the base member 368 and the carrier member 370, as shown by hidden lines in FIGS. 5-6. The elongated slots allow the carrier member 370 to be adjusted along the base member 368 to adjust the spacer 340. In use, the end plate 371 of the carrier member 370 engages the pressurizer 22 to provide proper pressurization. A sealing material, such as, for example, an elastomeric material, e.g., silicon rubber, foam, or the like, may be fixed to a face of the end plate 371 by an adhesive to provide a better seal between the end plate 371 and the pressurizer 22.

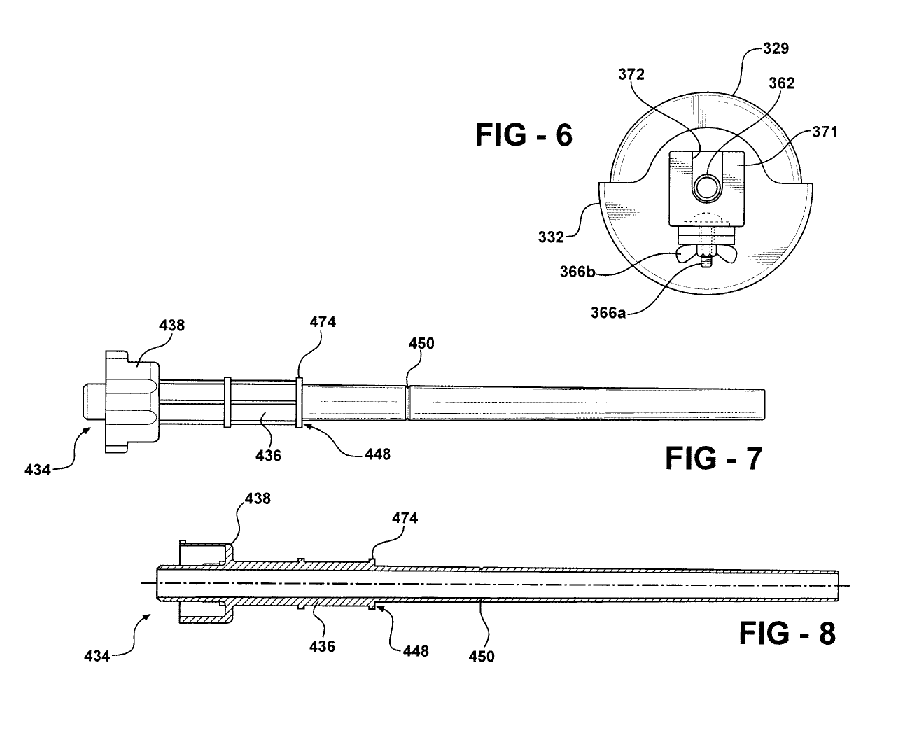

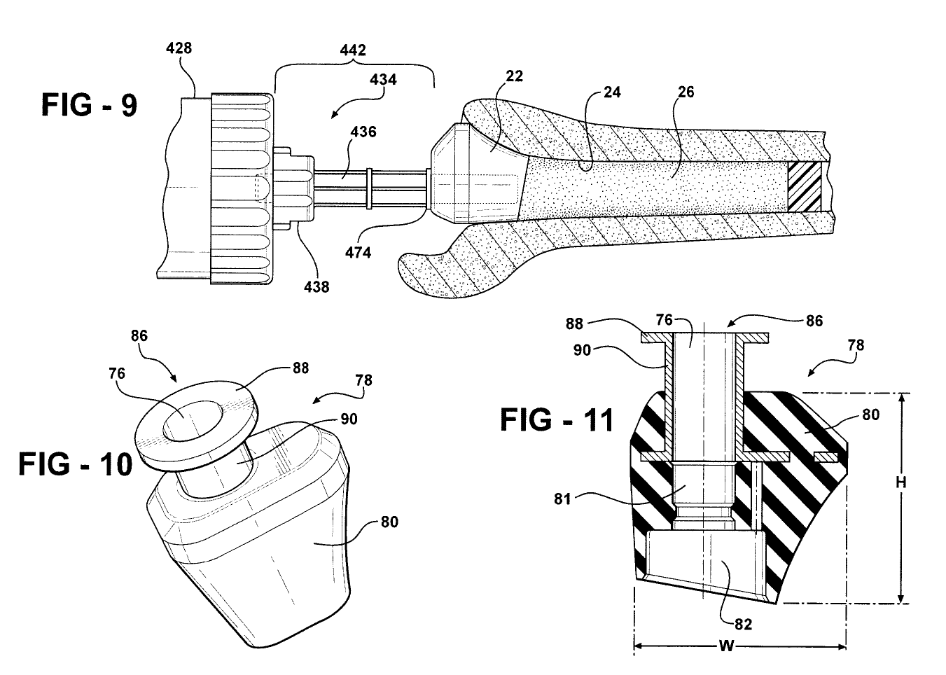

Referring to FIGS. 7-9, an alternative nozzle 434 is shown. In this embodiment, the same elements have been marked with the same numerals and like elements have been marked with similar numerals increased by 400. The nozzle 434 has an integral spacer 474 to apply pressure to the pressurizer 22. The nozzle 434 can be used to perform the retrograde technique to initially fill the bone canal 24 in the bone. The length of the nozzle 434 is shortened at the frangible section 450 and the pressurizer 22 is fitted on the shortened nozzle 434, as shown in FIG. 9. The pressurizer 22 is then inserted into the bone canal 24 of the bone. The face 448 of the integral spacer 474 is forced against the pressurizer 22. A sealing material, such as, for example, an elastomeric material, e.g., silicon rubber, foam, or the like, may be fixed to the face 448 of the integral spacer 474 by an adhesive to provide a better seal between the integral spacer 474 and the pressurizer 22. When pressure is applied to the pressurizer 22, the opening to the bone canal 24 is sealed and additional bone cement 26 is delivered through the pressurizer 22 into the opening to compact and pressurize the bone cement 26 in the bone canal 24.

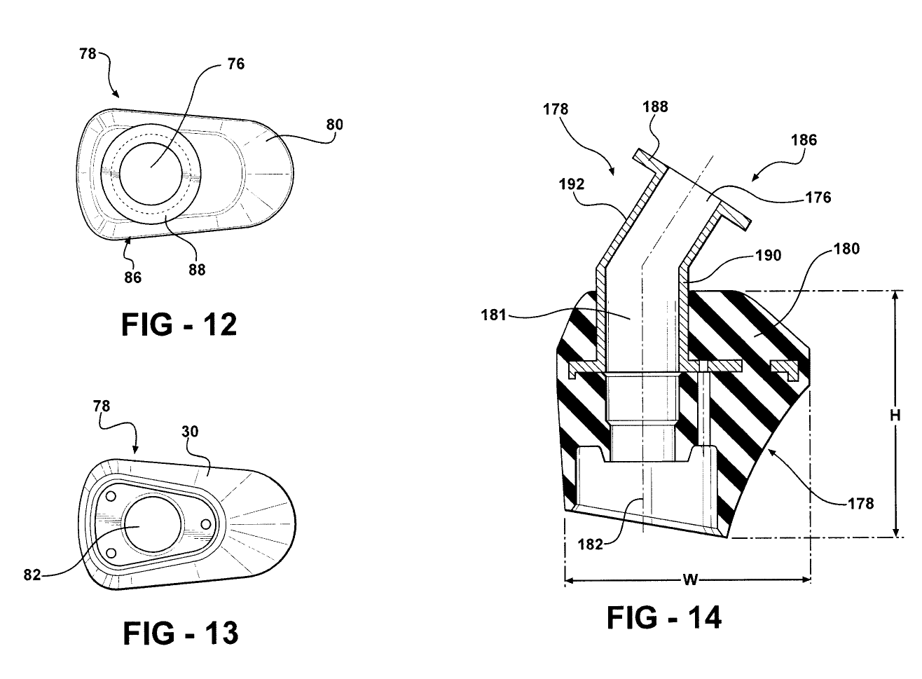

Referring to FIGS. 10-13, an alternative pressurizer 78 is shown. The alternative pressurizer 78 includes a body 80 preferably formed of a pliable material. The pliable material may be a durable material such as medical grade silicon or silicon rubber, or a softer material such as closed cell polyethylene foam or a closed cell silastic silicone foam. The body 80 includes an outer surface that seals against the bone in the area of the bone canal 24 similar to the pressurizer 22 shown in the previous embodiments. The body 80 defines a through-channel 81 for receiving the standard nozzle 362. The through-channel 81 extends to an outlet 82 for providing communication to the bone canal 24 during use.

The body 80 has a maximum height H and a maximum width W. The maximum height H is preferably less than 3 inches, more preferably less than 2 inches, and most preferably from 1 to 2 inches. The maximum width W is preferably less than 3 inches, more preferably less than 2 inches, and most preferably from 1 to 2 inches. The maximum width of the body 80 is generally measured transverse to the through-channel 81. By maintaining the maximum width below 3 inches, the alternative pressurizer 78 can be suited for use in MIS in which incisions are typically on the order of from 3 to 6 inches in length.

A neck 86 of rigid material extends from the body 80 to an end having a pressurizer spacer 88 spaced from the body 80 to provide the clearance distance between the container 28 and the body 80 during use to maintain a line-of-sight to the body 80 and the bone. The pressurizer spacer 88 is spaced from the body 80 at a distance of greater than 15 percent of the maximum width W, more preferably greater than 50 percent of the maximum width W, and most preferably greater than 100 percent of the maximum width W. The neck 86 includes an exterior wall 90 extending between the body 80 and the pressurizer spacer 88. The neck 86 defines an inlet 76 at the proximal end thereof for receiving the standard nozzle 362. It should be understood by those of ordinary skill in the art that the neck 86 can be integrally formed with the body 80 or formed separately and inserted into the through-channel 81. In the disclosed embodiment, the neck 86 would be formed from a harder, more rigid material than the body 80, such as, for example, a medical grade polymer or metal material, e.g., polycarbonate, polyethylene, stainless steel, or the like. In one embodiment, the body 80 is molded about the neck 86. The neck 86 has a diameter that is preferably less than 2 inches, more preferably less than 1.0 inches, and most preferably less than 0.5 inches. The diameter of the neck 86 is also preferably less than 75% of the maximum width W, more preferably less than 50% of the maximum width W, and most preferably less than 25% of the maximum width W.

The alternative pressurizer 78 is configured for use with a standard nozzle 362, such as in FIGS. 5-6, or with the nozzle 34, 134, 234, 434, of the embodiments that include the adjustable spacer 40, 140, 240 or the integral spacer 474. In use, the alternative pressurizer 78 is inserted into the bone canal 24 with the nozzle 34, 134, 234, 362, 434 placed in the through-channel 81 via the inlet 76 after the bone canal 24 is initially filled with the bone cement 26. The neck 86 provides the sufficient clearance distance between the end of the delivery system and the body 80 to provide the surgeon with adequate line-of-sight and a more visible work area to provide the necessary pressurization.

Referring to FIG. 14, a further embodiment of the alternative pressurizer 178 is illustrated. In this embodiment, the neck 186 includes an angled cylindrical wall portion 192 to provide an offset between the delivery system and the alternative pressurizer 178. This offset increases visibility to the surgeon to facilitate bone cement pressurization, which is particularly beneficial in an MIS procedure.

Obviously, many modifications and variations of the present invention are possible in light of the above teachings. The invention may be practiced otherwise than as specifically described within the scope of the appended claims.

What is claimed is:

1. A delivery system for use with a pressurizer to deliver a flowable material into an anatomical site during a medical procedure, said system comprising:
a container for holding the flowable material;
a nozzle having a proximal end operatively coupled to said container and a distal end spaced from said proximal end for conveying the flowable material to the anatomical site; and
a spacer adjustable along said nozzle between a plurality of adjustment positions for providing a variable clearance distance between said container and the pressurizer to maintain a line-of-sight to the pressurizer and the anatomical site during the medical procedure.

2. A delivery system as set forth in claim 1 including an adjustment mechanism operatively engaging said spacer for adjusting said spacer between said plurality of adjustment positions along said nozzle.

3. A delivery system as set forth in claim 2 wherein said adjustment mechanism includes a plurality of threads disposed along said nozzle and said spacer defines a threaded bore for engaging said plurality of threads.

4. A delivery system as set forth in claim 2 including a delivery gun for supporting said container when injecting the flowable material into the anatomical site.

5. A delivery system as set forth in claim 4 wherein said spacer includes a carrier member defining a U-shaped opening for receiving said nozzle and a base member adjustably interconnecting said delivery gun and said carrier member.

6. A delivery system as set forth in claim 5 wherein said adjustment mechanism includes a fastener adjustably coupling said carrier member to said base member.

7. A delivery system as set forth in claim 1 wherein said nozzle includes a flexible conduit.

8. A delivery system as set forth in claim 7 wherein said nozzle includes a nozzle body having a rigid section and said flexible conduit is detachably coupled to said rigid section.

9. A delivery system as set forth in claim 7 including a reinforcing coil embedded in said flexible conduit to reinforce said flexible conduit.

10. A delivery system as set forth in claim 1 wherein said nozzle includes a frangible section such that a user can break said nozzle at said frangible section to shorten said nozzle.

11. A delivery system as set forth in claim 1 wherein said nozzle includes a flared portion at said distal end for providing pressurization to the flowable material when delivering the flowable material to the anatomical site during the medical procedure.

12. A nozzle for use with a container holding a flowable material and a pressurizer to deliver the flowable material into an anatomical site during a medical procedure, said nozzle comprising:
a connector for operatively coupling said nozzle to the container;
a nozzle body fixed to said connector and extending to a distal end spaced from said connector for conveying the flowable material to the anatomical site; and
a spacer adjustable along said nozzle body between a plurality of adjustment positions for providing a variable clearance distance between the container and the pressurizer to maintain a line-of-sight to the pressurizer and the anatomical site during the medical procedure.

13. A nozzle as set forth in claim 12 including an adjustment mechanism operatively engaging said spacer for adjusting said spacer between said plurality of adjustment positions along said nozzle body.

14. A nozzle as set forth in claim 13 wherein said adjustment mechanism includes a plurality of threads disposed along said nozzle body and said spacer defines a threaded bore for engaging said plurality of threads.

15. A nozzle as set forth in claim 12 wherein said nozzle body includes a flexible conduit.

16. A nozzle as set forth in claim 15 wherein said nozzle body has a rigid section and said flexible conduit is detachably coupled to said rigid section.

17. A nozzle as set forth in claim 16 including a reinforcing coil embedded in said flexible conduit to reinforce said flexible conduit.

18. A method of delivering a flowable material to an anatomical site under pressure during a medical procedure using a delivery system having a container for holding the flowable material and a nozzle with a spacer engaging a pressurizer, said method comprising the steps of:
sealing the pressurizer at the anatomical site by pressing the pressurizer against the anatomical site with the spacer of the nozzle;
delivering the flowable material to the anatomical site under pressure with the pressurizer sealed to the anatomical site and the spacer pressed against the pressurizer; and
adjusting the spacer of the nozzle between a plurality of adjustment positions prior to sealing the pressurizer at the anatomical site to provide a predetermined clearance distance between the container and the pressurizer and maintain a line-of-sight to the pressurizer and the anatomical site during the medical procedure.

19. A method as set forth in claim 18 including delivering a first amount of the flowable material to the anatomical site through the nozzle prior to sealing the pressurizer at the anatomical site.

20. A pressurizer for use with a delivery system having a container for holding a flowable material and a nozzle operatively coupled to the container to convey the flowable material into an anatomical site under pressure during a medical procedure, said pressurizer comprising:
a body of pliable material defining a channel for receiving the nozzle and an outlet for providing communication between the nozzle and the anatomical site, said body having an outer surface for sealing against the anatomical site;
said body having a maximum width measured generally transverse to the channel, said width being less than 3 inches to facilitate insertion of said body into a minimally invasive incision; and
a neck of rigid material extending from said body to an end spaced from said body for providing a clearance distance between the container and said body to maintain a line-of-sight to said body and the anatomical site after inserting said pressurizer into the minimally invasive incision.
